(19) United States
(12) Patent Application Publication
(10) Pub. No.: US 2023/0213129 A1
(43) Pub. Date: Jul. 6, 2023

(54) ASEPTIC COUPLING ASSEMBLY AND METHOD OF ASEPTIC COUPLING

(71) Applicant: ENTEGRIS, INC., Billerica, MA (US)

(72) Inventors: Jacob Peabody, Roxbury, MA (US); Nicholas Coscia, Lexington, MA (US); David Schroeder, Bloomington, MN (US); Tate Vangsgard, Oakdale, MN (US)

(21) Appl. No.: 18/090,791

(22) Filed: Dec. 29, 2022

Related U.S. Application Data
(60) Provisional application No. 63/297,148, filed on Jan. 6, 2022.

(51) Int. Cl. F16L 37/35; F16L 37/084

(52) U.S. Cl. CPC F16L 37/35; F16L 37/0841; F16L 2201/44

(57) ABSTRACT

An aseptic coupling assembly, includes a first connector and a second connector. The first connector includes a first housing, a first valve member, and a first spring that biases the first valve member towards the second end of the first housing to a first closed position. The second connector includes a second housing, a second valve member, and a second spring that biases the second valve member towards the second end of the second housing to a second closed position. The first valve member is slidable away from the first closed position within the first housing to compress the first spring and the second valve member is slidable away from the second closed position within the second housing to compress the second spring. The coupling assembly further includes a seal that prevents fluid from contacting the first spring and the second spring.

### PRIORITY

This disclosure claims priority to U.S. provisional patent No. 63/297,148 with a filing date of Jan. 6, 2022, which is incorporated in its entirety herein.

### FIELD

This disclosure relates generally to fluid connection assemblies for transferring a fluid. More specifically, this disclosure is directed to coupling and coupling assemblies used in aseptically coupling of a low-temperature storage container.

### BACKGROUND

Chemical and/or biological processes can utilize or produce process materials that are stored within storage containers containing pharmaceutical or biological fluids, such as, bioprocess bags, and the like. Tubing or other types of coupling may be utilized to supply the process material and/or reactants into to the storage container. The process materials may need to be frozen or otherwise kept at low temperatures within the storage container. Tubing or other types of coupling may then be utilized to remove the process material from the storage container, but the tubing or other types of coupling need to maintain a sterile, non-contaminated environment.

### SUMMARY

In an embodiment, an aseptic coupling assembly is provided. The aseptic coupling assembly includes a first connector including a first fluid passage extending through the first connector, the first fluid passage including an opening, and a second connector including a second fluid passage extending through the second connector, the second fluid passage including an opening. The first connector includes a first housing having a first end and a second end, a first valve member provided to be slidable within the first housing, and a first spring that biases the first valve member towards the second end of the first housing to a first closed position. The second connector includes a second housing having a first end and a second end, a second valve member provided to be slidable within the second housing, and a second spring that biases the second valve member towards the second end of the second housing to a second closed position. The first valve member and the second valve member include an internal passageway fluidly connected to the first fluid passage and the second fluid passage, respectively. The first connector and the second connector are configured to be coupled together, in which the first valve member is slidable away from the first closed position within the first housing to compress the first spring and the second valve member is slidable away from the second closed position within the second housing to compress the second spring so that the first fluid passage and the second fluid passage are fluidly connected. The aseptic coupling assembly further includes a seal that is configured such that fluid flowing through the coupling assembly is prevented from contacting the first spring and the second spring.

In an embodiment, a method of aseptically connecting a fluid storage container includes coupling a first connector to a second connector. A first fluid passage extending through the first connector and a second fluid passage extending through the second connector are fluidly connected. The first fluid passage and the second fluidly passage each include an opening, in which the first connector includes a first housing having a first end and a second end, a first valve member provided to be slidable within the first housing, and a first spring that biases the first valve member towards the second end of the first housing to a first closed position. The second connector includes a second housing having a first end and a second end, a second valve member provided to be slidable within the second housing, and a second spring that biases the second valve member towards the second end of the second housing to a second closed position. The method further includes coupling the first connector to the second connector which includes engaging a first surface of the first valve member and a first surface of the second valve member such that the first valve member is slid away from the first closed position within the first housing to compress the first spring and the second valve member is slid away from the second closed position within the second housing to compress the second spring so that the first fluid passage and the second fluid passage are fluidly connected; and preventing fluid flowing through the first connector and the second connector from contacting the first spring and the second spring, respectively, by isolating the first spring and the second spring from the fluid.

### DRAWINGS

FIG. 1 is a front perspective view of an embodiment of an aseptic coupling assembly.

FIG. 2 is an exploded view of the aseptic coupling assembly according to FIG. 1, according to an embodiment.

FIG. 3 is a sectional view of the aseptic coupling assembly according to FIG. 1, according to an embodiment, in a coupled state.

FIG. 4 is a sectional view of the aseptic coupling assembly according to FIG. 1, according to an embodiment, prior to disconnecting the connectors.

FIG. 5 is a sectional view of the aseptic coupling assembly according to FIG. 1, according to an embodiment, in which the connectors are being disconnected.

FIG. 6 is a sectional view of the aseptic coupling assembly according to FIG. 1, according to an embodiment, in which the connectors are disconnected.

FIGS. 7A, 7B, 7C are perspective and cross-sectional views of a latch mechanism, according to an embodiment.

FIG. 8 is a schematic diagram of an embodiment of a storage container aseptically connected to a processing device with an assembled aseptic coupling assembly.

Like numbers represent like features.

### DETAILED DESCRIPTION

This disclosure relates generally to fluid connection assemblies for transferring a fluid. More specifically, the disclosure relates to an aseptic coupling and coupling assemblies used in aseptically coupling and transferring, e.g., filling and removal, of fluids from storage containers to avoid contamination. The term aseptic as used herein is at least related to creating a barrier or fluid path that remains unbroken to keep the fluid substantially free of contaminants from an external environment to maintain the sterility of the internal content and/or components. While the fluids related to chemical and/or biological processes are discussed below, it is appreciated that such discussion is not intended to limit the scope of the invention, but provided as embodiments thereof.

Some chemical and/or biological processes utilize or produce process materials that are stored within storage containers, such as bags, containing pharmaceutical or biological fluids. The pharmaceutical or biological fluids may also need to be frozen or otherwise kept at low temperatures within the storage container. It is appreciated that a fluid includes, but is not limited to, a substance that flows or deforms when a shear stress is applied. A fluid can include, for example, a liquid.

In some cases, the pharmaceutical or biological fluids in the storage container needs to be processed aseptically during the supply, storage, testing, and/or removal of the pharmaceutical or biological fluids. That is, the pharmaceutical or biological fluids are provided in a sterilized environment and are transferrable from the storage container under sterile conditions. For example, films, caps, valves, or similar sealing devices that maintain a hermetic seal can be attached and/or coupled to the storage container and/or any processing equipment for the aseptic connection/disconnection of the storage container to any processing equipment. The film, caps, valves, or similar sealing devices can then be punctured, removed, and/or turned to allow the transfer of the sterile pharmaceutical or biological fluids to the processing equipment when the storage container is coupled to the processing equipment and disconnected using films, caps, automatic shut-off valves, or similar devices that allow an aseptic disconnect functionality.

It was observed, however, that such prior aseptic assemblies were not suitable for use at temperatures as low as −196° C. or lower, in which the ultra-cold temperature can cause a leakage in the aseptic assembly that allowed ingress of the cryogenic fluid, e.g., liquid nitrogen or similar, into the storage container or egress of the contents thereof. Thus, causing failure of the aseptic assembly and breaking sterility of the system. Moreover, the prior spring loaded aseptic assemblies while providing a sterile barrier, has the fluid contact the metal springs. Thus, the fluid is in contact with a possible source of contamination, e.g., from metal leaching from the spring, and, therefore, the fluid should not contact the metal spring.

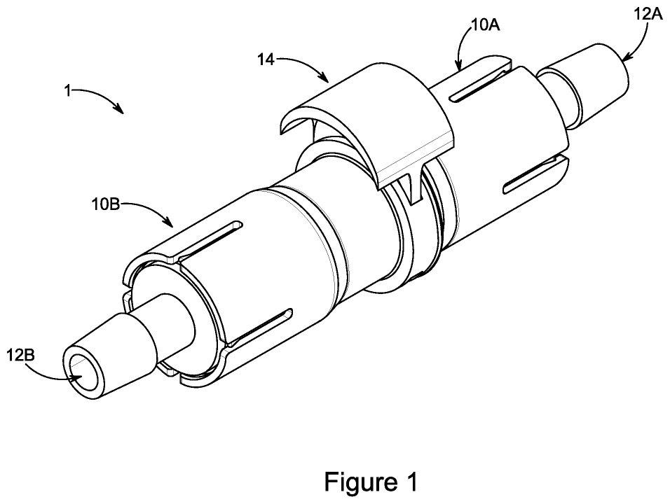

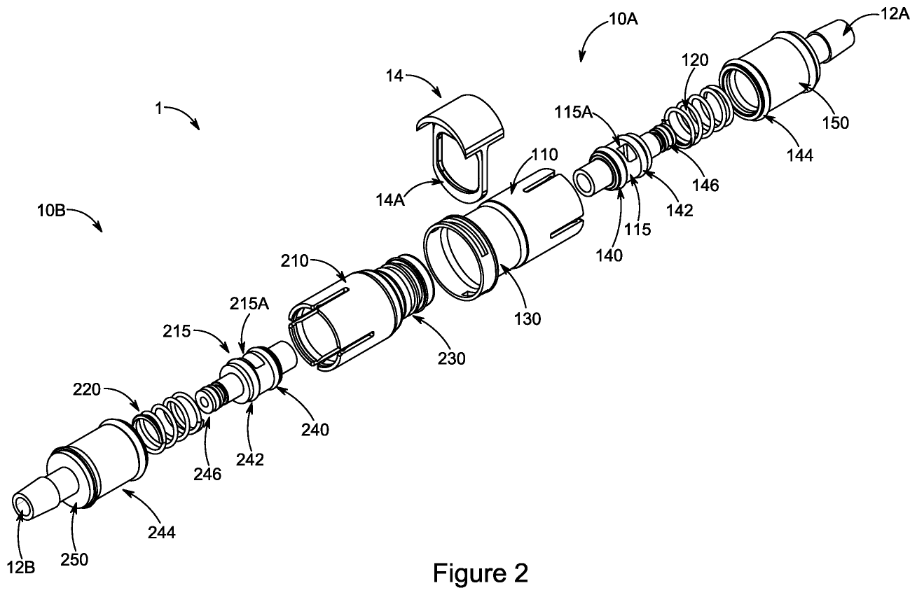

FIGS. 1 and 2 are perspective views of an embodiment of an aseptic coupling assembly 1 for overcoming the deficiencies, as discussed above. The coupling assembly 1 is configured to connect and disconnect from a storage container for the aseptic transferring of the pharmaceutical or biological fluids that can be used in temperatures as low as −196° C. It is appreciated that while the aseptic coupling assembly 1 can be used at cryogenic temperatures, the aseptic coupling assembly 1 can also be used at other temperatures, such as −80° C., 0° C., room temperature, or the like, while maintaining the benefits of the present disclosure.

In an embodiment, the aseptic coupling assembly 1 can be a single-use aseptic disconnector or multi-use connector that is assembled in-line with tubing and/or the storage container. The coupling assembly 1 includes a first connector 10A and a second connector 10B that are configured to couple together to form a sealed fluid connection and a latch mechanism 14 for disconnecting the first connector 10A and second connector 10B. The first connector 10A includes a first fluid passage 12A that extends through the first connector 10A from an opening. The second connector 10B includes a second fluid passage 12B that extends through the second connector 10B from another opening. When the first connector 10A is coupled to the second connector 10B, the first fluid passage 12A and the second fluid passage 12B are joined to form a sealed fluid connection that extends through the coupled connectors 10A, 10B connecting the openings.

As seen in FIG. 2, the first connector 10A includes a first housing 110 having a first end and a second end, a first valve member 115 that is slidable within the first housing 110, and a first spring 120 that is configured to bias the first valve member 115 towards the second end of the first housing to a first closed position, as discussed further below. The second connector 10B includes a second housing 210 having a first end and a second end, a second valve member 215 that is slidable within the second housing 210, and a second spring 220 that is configured to bias the second valve member 215 towards the second end of the second housing to a second closed position. The first ends of the housings 110, 210 form a first portion of the housings 110, 210 and the second ends of the housings 110, 210 form a second portion of the housings 110, 210, in which the second portions of the housings 110, 210 have a diameter smaller than a diameter of the first portions. In an embodiment, between the first portion and the second portion of the housings 110, 210, inclined portions can be provided as the diameter decreases between the first portion and the second portion. The inclined portions can be at an angle between 10 degrees and 45 degrees, and preferably about 20 degrees. The inclined portions can be provided along the housing at positions corresponding to the valve members and/or connection portions, as discussed below. It is also appreciated that in some embodiments, the diameter of the first portion and the second portion of the housing is not decreased along an external surface, but the diameter is decreased internally of the housing.

The first connector 10A and the second connector 10B are configured to be coupled together. For example, in an embodiment, the second portion of the housing 210 can include a mating end 230 that is receivable in a receiving end 130 of the first housing 110 formed in the second portion of the first housing 110. At least one sealing member can be provided between the receiving end 130 of the first connector 10A and the mating end 230 of the second connector 10B for maintaining a fluid-tight seal, in which the sealing member can be a flexible and/or compressible material, such as, silicone and ethylene-vinyl acetate (EVA), or the like. It is appreciated that the first connector 10A and the second connector 10B can also be connected using different connection arrangements. For example, in an embodiment, the mating end 230 can be provided on the first housing 110 of the first connector 10A and the receiving end can be provided on the second housing 210 of the second connector 10B. Additionally, it is appreciated that the connector can use genderless coupling arrangements, threaded connections, push-to-connect connections, or the like.

As discussed above, the first valve member 115 and the second valve member 215 are provided to be slidable at least in the respective housings of the first connector 10A and the second connector 10B. In an embodiment, the first valve member 115 and the second valve member 215 each include an internal passageway that are connected to the fluid passage of the respective connector. The internal passageway can be a bore through the respective valve member along a first portion of the valve member. The internal passageway is fluidly connected to a slot 115A, 215A provided in a second portion of the valve member. The slot 115A, 215A is provided to fluidly connect the internal passageway to a space external to the valve member within the housing 110, 210. In an embodiment, the slot 115A, 215A can be provided on opposite sides of the valve member having a rectangular opening, circular opening, ovular opening, or the like, can be provided as multiple slots provided around a circumference of the valve member, or similar structure that allows fluid communication of the internal passageway to the outer space, or combinations thereof.

Springs 120, 220 are provided in the housings 110, 210 to bias the valve members 115, 215 to the closed position towards the second ends of the housings 110, 210. In an embodiment, the springs 120, 220 can be provided externally of the first fluid passage 12A and the second fluid passage 12B so that the fluid flows through the internal passageway. The springs 120, 220 can be metal springs or the like that are able to provide a spring force sufficient to separate the first connector 10A and the second connector 10B.

The coupling assembly 1 further includes a seal that is a structure for guiding the flow of fluid through the coupling assembly 1 when assembled. For example, in some embodiments, the seal can include a plurality of sealing members that are provided between the first housing 110 and the first valve member 115 and the second housing 210 and the second valve member 215 to prevent the fluid flowing through the coupling assembly 1 from contacting the first spring 120 and the second spring 220. For example, in an embodiment, sealing members 140, 142, 240, 242 can be provided around the slots 115A and 215A, e.g., along a longitudinal axis, of the first valve member 115 and the second valve member 215 for sealing and guiding fluid flow between the valve member 115, 215 and the respective housing 110, 210.

In some embodiments, sealing members are also provided to prevent the ingress and egress of the pharmaceutical or biological fluid from the coupling assembly 1. For example, in an embodiment, sealing members 144, 244 can be provided on connection portions 150, 250 of each connector 10A, 10B, if provided, for sealing the connection portion 150, 250 and the housing 110, 210, respectively. In an embodiment in which the housings 110, 220 include the inclined portion, the inclined portion provided a structure in which, during assembly of the connectors 10A, 10B, the force required to assemble the connectors is reduced when connecting the connection portions 150, 250 to the housings 110, 210 at least in part due to the incline of the inclined portion. In an embodiment, sealing member 146, 246 can also be provided on the valve member 115, 215 around the internal passageway to create a fluid tight seal between the valve member 115, 215 and the connection portion 150, 250. It is appreciated that the sealing members, as discussed herein and above, can be an O-ring or gasket formed from, but not limited to, at least one of silicone, a fluoropolymer, ethylene-vinyl acetate (EVA), a thermoplastic, or other biocompatible flexible and/or compressible material for sealing that is relatively inert, e.g., does not leach or significantly absorb the pharmaceutical or biological fluid, and non-reactive, or a combination thereof. The sealing member can also include quad-rings, or a thermoplastic material or other flexible and/or compressible material for sealing that is melt processed to fill grooves in the respective members with the thermoplastic or flexible material, and/or formed from any of the above materials. It is also appreciated that the seal can include other structures that guide the fluid flow through the coupling assembly 1. For example, in an embodiment, the seal can include the connection portions 150, 250 that are melt-processed with the housing 110, 210, to form a fluid-tight seal using welding or bonding techniques such as, but not limited to, heat bonding, impulse welding, laser welding, ultrasonic welding, platen welding, or similar fusion bonding/melt welding techniques. In other embodiments, the seal can include an enclosed cavity that houses the springs 120, 220, in which the enclosed cavity is compressible or uses magnets for moving the valve members 115, 215.

The latch mechanism 14 is provided for securing the first connector 10A and the second connector 10B together and disconnecting the same. In an embodiment, the latch mechanism 14 can be provided through a slot in the first housing 110 and configured to engage with the second connector 10B. In some embodiments, the latch mechanism 14 can include a projection 14A for engaging an outer surface of the second housing 210, for example, a groove provided near or adjacent to the mating end 230. A biasing member (not shown) can be provided for biasing the latch mechanism in a retaining position so that the projection 14A remains engaged with the outer surface of the second housing 210. In an embodiment, the biasing member can be a spring that biases the latch mechanism 14 upwards and away from the coupling assembly 1, so that the projection 14A remains engaged with the outer surface of the second housing 210. In another embodiment, the biasing member can be a spring that biases the latch mechanism 14 in a displacement position, in which the latch mechanism 14 is engageable with a protrusion on the outer surface of the second housing 210, e.g., hook, so that when the hook of the latch mechanism 14 is disengaged from the protrusion, the spring biases the latch mechanism 14 for uncoupling and/or separating the connectors. In other embodiments, the biasing member can include bumps or protuberances on at least one of the latch mechanism or the outer surface of the second housing 210 that engage dimples or divots in the other respective member. Thus, the latch mechanism 14 can use frictional and/or tensional force for retaining the second connector 10B to the first connector 10A for connecting the same. In other embodiments, the latch mechanism 14 can include other designs for connecting the first connector 10A and the second connector 10B together. For example, in an embodiment, the latch mechanism 14 does not include the biasing member, but instead can include the bumps or protuberances that engage dimples or divots, use a twist-action latching assembly, or the like for connecting the housings together. It is appreciated that while the latch mechanism 14 has been discussed with respect to being on the first connector 10A, in some embodiments, the latch mechanism 14 can be included on the second connector 10B, in which the first connector 10A has the corresponding structure for connecting the connectors together and/or any combination thereof.

In some embodiments, the coupling assembly 1 can further include connection portions 150, 250 for each connector 10A, 10B for connecting to a storage container and/or processing equipment for the supply, storage, testing, processing, and/or removal of the pharmaceutical or biological fluids. The connection portion 150, 250 can include a housing for receiving the spring 120, 220 and the valve member 115, 215 and a connection fitting, such as a hose barbed connection. The connection portion 150, 250 can include an internal chamber for housing the spring 120, 220, in which the internal chamber is provided concentrically around the fluid passages 12A, 12B and the internal passageway of the valves 115, 215. The connection fitting can be attached to tubing via the hose barbed connection or can be overmolded to a fitment such as the boat fitment on the storage container, such as used in a bag assembly. It is appreciated that the connection fitting can be any type of connection including, but not limited to, threaded fittings, sanitary flanges, compression fittings, luer fittings, luer-lock fittings, and the like that can be connected or molded or overmolded to the storage container, tubing, or combinations thereof. In an embodiment, the connection portion 150, 250 can be interchangeable with its respective housing 110, 210. For example, in an embodiment, the connection portion 150, 250 can be press-fit into the first housing 110 or the second housing 210, in which a sealing member provides a fluid-tight seal between the connection portion 150, 250 and the respective housing 110, 210. In some embodiments, the first housing 110 and/or the second housing 210 includes retaining features, such as, for example, slots, tabs, flanges, detents, hooks, or any other suitable structures for mechanical engagement with a surface of the connection portion 150, 250 for retaining the same. It is appreciated that the connection portion 150, 250 can also be connected (or disconnected) from the respective housing 110, 210 using other types of connection including, but not limited to, threaded fittings, compression fittings, or the like.

In some embodiments, the aseptic coupling assembly 1 and/or the components thereof can be formed from thermoplastic polymer selected from the group consisting of fluoropolymers, polyurethanes, vulcanizate, flexible polyvinyl chloride (PVC), thermoplastic elastomer (TPE), high-density polyethylene (HDPE), ethylene-vinyl acetate (EVA), co-polymer/polyolefin, high-impact polystyrene (HIPS), polypropylene (PP), acrylonitrile butadiene styrene (ABS), polytetrafluoroethylene (PTFE, or ETFE), or blend of the same, or a thermoset elastomer, such as liquid silicone rubber (LSR) or blend of the same. Thus, the connectors are made from a material that is able to be used at cryogenic temperatures, e.g., −196° C. The connector can also be made of material that is relatively inert, e.g., does not leach or significantly absorb the pharmaceutical or biological fluid, and non-reactive with the pharmaceutical or biological fluid.

Accordingly, the aseptic coupling assembly 1, as discussed above, is configured to be stored at frozen temperatures (e.g., temperatures of less than 0° C.). In an embodiment, the aseptic coupling assembly 1 is configured to be stored at cryogenic temperatures of −150° C. or lower. In an embodiment, the aseptic coupling assembly 1 is configured to be stored at cryogenic temperatures of −196° C. or lower. In an embodiment, the aseptic coupling assembly 1 can also be heated back to ambient temperature without having any substantial deformation. Substantial deformation includes, for example, visible cracking in the material, a shrinkage or expansion relative to its original shape at ambient temperature that can interfere with the connection(s) or adversely affect sealing of said connection(s). Temperature retraction testing can be performed according to ASTM D1329, ISO 2921, or any other suitable testing methodology for determining suitable retraction properties of the material at temperatures that may be used. Brittleness testing can be performed according to ASTM D2137, ISO 28702, or any other suitable testing methodology for determining resistance to cracking at temperatures that may be used.

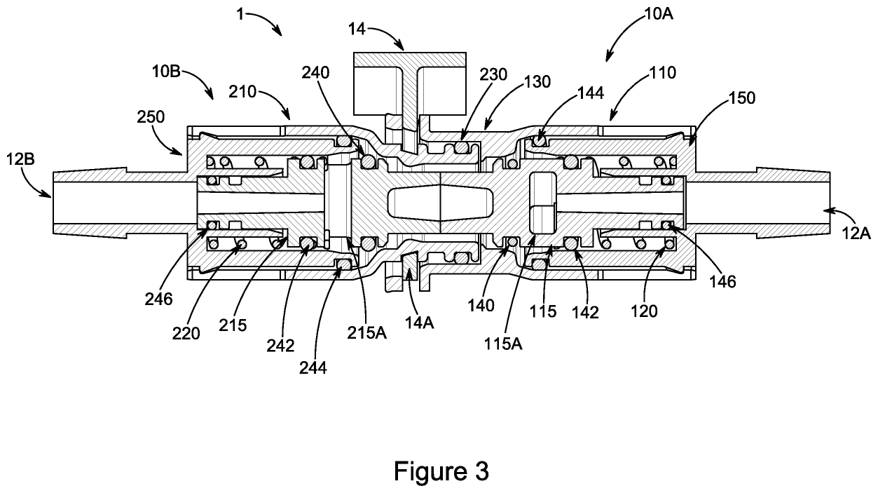

The operation and connection/disconnection of the aseptic coupling assembly 1 will be discussed while referencing FIGS. 3-6 below.

As seen in FIG. 3, the first connector 10A and the second connector 10B of the coupling assembly 1 are coupled together to form an aseptically sealed fluid connection from a source, e.g., bioprocessing bag, to a destination, e.g., processing equipment for processing a pharmaceutical or biological fluid. For example, in an embodiment, the coupling assembly 1 can be connected by directing the latch mechanism 14 downwardly towards the coupling assembly 1 so that the latch projection 14A does not contact the second housing 210 of the second connector 10B and inserting the mating end 230 of the second connector 10B into the receiving end 130 of the first connector 10A. The mating of the first connector 10A and the 10B creates an engagement between surfaces of the first valve member 115 and the second valve member 215 so that the first valve member 115 is slid from the first closed position, e.g., the position in which the sealing member 140 contacts the inclined portion of the first housing 110 sealing the first connector 10A, which compresses the first spring 120, and the second valve member 215 is slid from the second closed position, e.g., the position in which the sealing member 240 contacts the inclined portion of the second housing 210 sealing the second connector 10B, which compresses the second spring 220, so that first fluid passage 12A and the second fluid passage 12B are fluidly connected, as discussed below. It is appreciated that the sliding of the first valve member 115 and the second valve member 215 can be under equal force, e.g., the same spring force to compress equally, or more force can be needed to open the first connector 10A, e.g., unequal spring force. It is also appreciated that the ramping distance of the inclined portion can be a distance in which the movement of the valve members 115, 215 does not allow fluid to flow until the connectors 10A, 10B are coupled together, e.g., the fluid is shut off prior to any exposure to the environment. It is appreciated that in an embodiment, a film or cap or cover can be provided on ends of the first connector 10A and the second connector 10B, which can be removed prior to connecting the same, to maintain the sterility of the system.

In some embodiments, when the first connector 10A and the second connector 10B are coupled together, the first fluid passage 12A and the second fluid passage 12B are connected so that the fluid can flow through the first fluid passage 12A, through the internal passageway of the first valve member 115, and out of the first valve member 115 through the slot 115A to a space external of the first valve member 115 within the first housing 110. The fluid can then flow around the sealing member 140 and then flow externally of the valve members 115, 215 in the mating end 230 of the second housing 210. The fluid can then flow around the sealing member 240 and enter the slot 215A of the second valve member 215 to be fluidly connected with the internal passageway of the second valve member 215 and the second fluid passage 12B.

The latch mechanism 14 can then be used for securing and/or retaining the second connector 10B and the first connector 10A. For example, in an embodiment, the latch mechanism 14 is biased upward, e.g., away, from the coupling assembly 1 so that the latch projection 14A engages the outer surface of the second housing 210, e.g., a groove, to retain the second connector 10B to the first connector 10A. For example, in some embodiments, a spring can be used to bias the latch 14 in a connected position when the latch mechanism 14 is released. It is also appreciated that other biasing members can be used for retaining the latch 14 in the connected position. For example, in some embodiments, the biasing member can include bumps or dimples that must be overcome to latch and/or unlatch the latch 14 from the disconnected and/or connected position to the opposite position.

FIG. 3 further shows that the aseptic coupling assembly 1 is configured so that that fluid flowing through the coupling assembly is prevented from contacting the first spring and the second spring 120, 220. For example, in an embodiment, a seal includes sealing members 142, 242 that fluidly seal the internal chamber in the connection portions 150, 250, in which the springs 120, 220 are provided externally of the first fluid passage 12A and the second fluid passage 12B. Thus, as the valve members 115, 215 are slid from the closed position to an open position, so that fluid flows through the coupling assembly 1, the sealing members 142, 242 prevent the fluid from contacting the springs 120, 220, since the fluid flows through the fluid passages 12A, 12B, through the internal passageway of the valve members 115, 215 and externally of the valve members 115, 215 away from the springs 120, 220, e.g., fluid flows to path of least resistance.

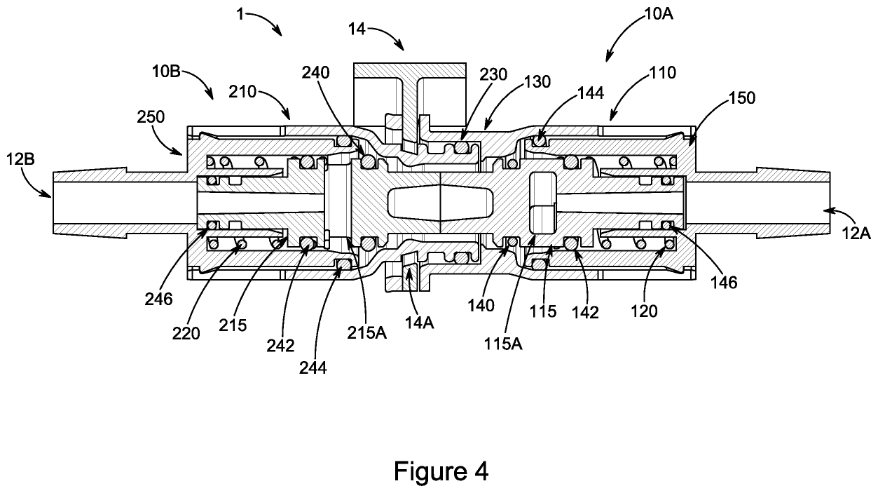

The aseptic disconnect of the aseptic coupling assembly 1 is discussed with reference to FIGS. 4-6.

FIG. 4 shows that in order to disconnect the second connector 10B from the first connector 10A, the latch mechanism 14 is pushed downwardly so that the latch projection 14A is disengaged from the outer surface of the second housing 210, which allows the disengagement of the mating end 230 of the second connector 10B from the receiving end 130 of the first connector 10A.

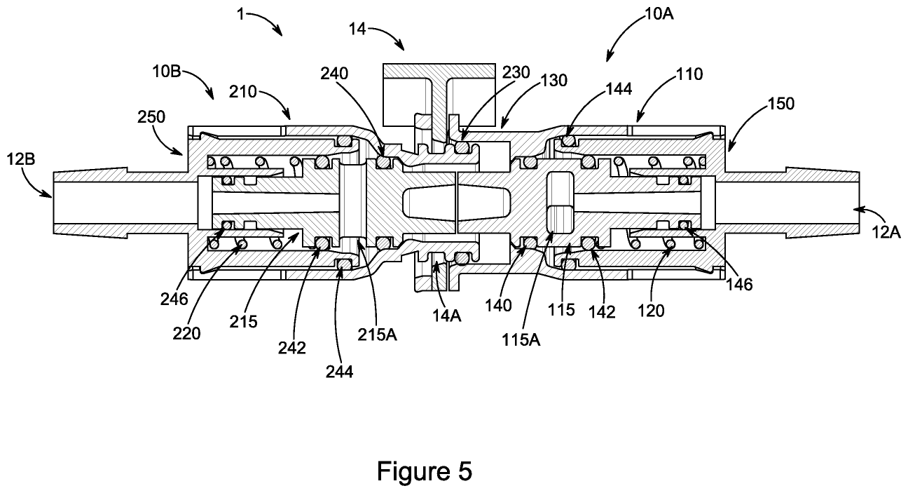

As seen in FIG. 5, as the latch mechanism 14 is pushed downwardly, springs 120, 220 begin to bias the respective valve member 115, 215 so that the second connector 10B is separated from the first connector 10A, and the valve members 115, 215 are biased to the closed position. That is, no external force is required by a user to pull the connectors apart and the connectors 10A, 10B are self-sealing of the fluid flow. For example, in an embodiment, the sealing members 140, 240 on the valve members 115, 215 are biased towards the second end of the housings 110, 210 so that the sealing members 140, 240 engage the internal surface of the housings 110, 210. It is appreciated that the internal surface of the housings 110, 210 can be provided as a number of different structures. In some embodiments, the internal surface of the housings 110, 210 include the inclined portion so that as the sealing members 140, 240 are biased to the closed position, the fluid flowing around the valve members 115, 215 is gradually stopped, e.g., at least due in part to the gradual compression of the sealing members, which can aid in limiting or preventing the egress of the pharmaceutical or biological fluid to an external environment. It is appreciated that in some embodiments, the first connector 10A and the second connector 10B can be separated using an external force, e.g., user applied force, for pulling the connectors apart, so that less spring force is required to seal the connectors 10A, 10B.

It is appreciated that similar to the discussion with respect to FIG. 3 above, the aseptic coupling assembly is configured so that that fluid flowing through the coupling assembly is prevented from contacting the first spring and the second spring 120, 220. For example, in an embodiment, as the valves 115, 215 are biased to the closed position, the sealing members 142, 242 maintain a fluidic seal of the internal chamber of the connection portions 150, 250. Thus, as the valve members 115, 215 are slid to the closed position, the sealing members 142, 242 prevent the fluid from contacting the springs 120, 220, since the fluid flows through the fluid passages 12A, 12B, through the internal passageway of the valve members 115, 215 and externally of the valve members 115, 215 and is sealed by sealing members 140, 142, 240, 242 to prevent any further flow of fluid.

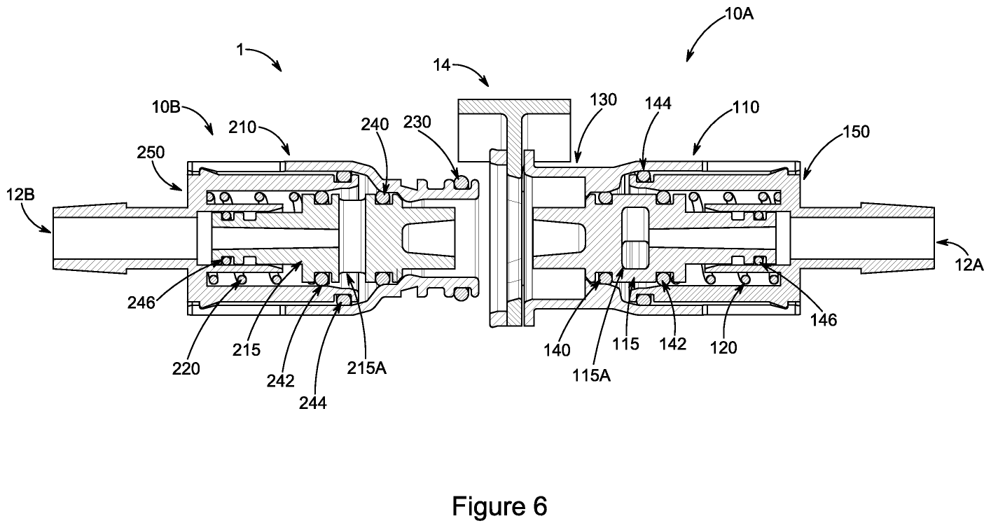

FIG. 6 illustrates the disconnection of the second connector 10B from the first connector 10A. After the valve members 115, 215 are biased to the closed position by springs 120, 220 so that fluid stops flowing through the connectors 10A, 10B, the second connector 10B can be further separated from the first connector 10A. As seen in FIG. 6, the housings 110, 210 have a length at the second ends that is longer than the valve members 115, 215, so that as the connectors 10A and 10B are disconnected, valve members 115, 215 are offset with respect to the opened end of the housing 110, 210. That is, the offset of the valve members 115, 215 forms a lip portion of the housing that extends past the valve members 115, 215 so that the lip portion can be used to protect the valve members 115, 215 from accidently being pushed in and/or contacted, or moved away, which can cause contamination and/or egress of the pharmaceutical or biological fluid, and can allow the covering of the connectors 10A, 10B using a film or cap or cover.

In view of at least the discussion above with respect to the structure and operation of the aseptic coupling assembly 1, it is appreciated that the aseptic coupling assembly 1 is not only suitable for use in temperatures as low as cryogenic temperatures of −196° C. or lower, but also provides a sterile barrier in which the fluid does not contact the metal springs, which avoids a possible source of contamination, e.g., from metal leaching from the spring.

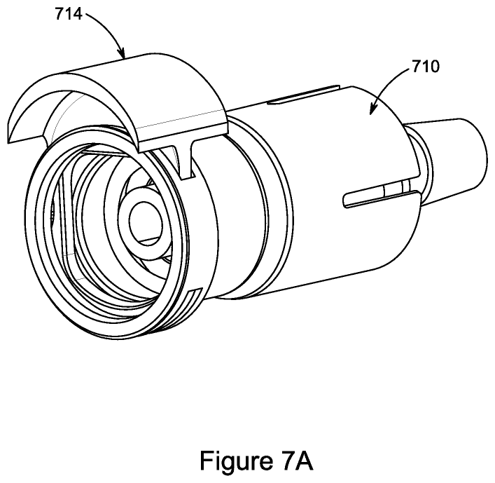

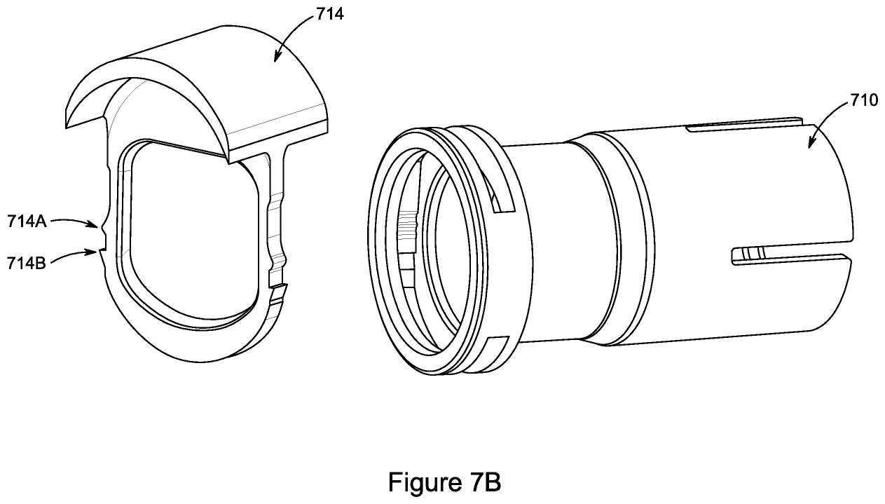

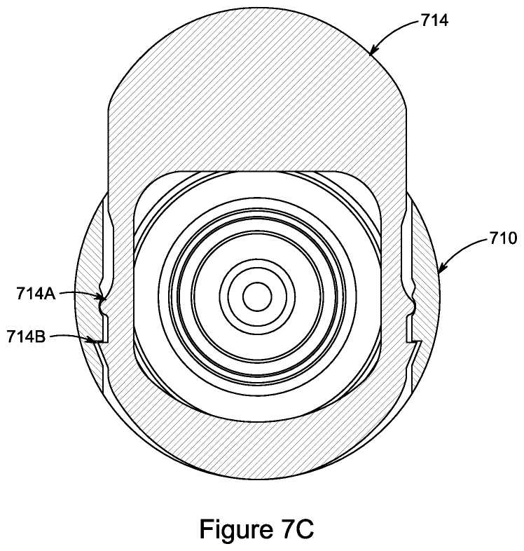

FIGS. 7A, 7B, 7C illustrate a coupling assembly that includes a latch mechanism 714 that has biasing members that include bumps or protuberances 714A, 714B, in an embodiment, as discussed above. The coupling assembly according to this embodiment includes the same elements as the coupling assembly 1, as discussed above, but is discussed with respect to the latch mechanism and first housing to clarify the description.

FIGS. 7A and 7B show perspective views of the latch mechanism 714 and first housing 710, in which the latch mechanism 714 is provided for securing the first connector 10A and the second connector 10B together and disconnecting the same. The latch mechanism 714 can be provided through a slot in the first housing 710 and configured to engage with the second connector 10B, as discussed above. The latch mechanism 714 includes a biasing member for biasing the latch mechanism in a retaining position so that the projection of the latch mechanism 714 remains engaged with the outer surface of the second housing (not shown). The biasing member is also provided to allow the disengaging from the retaining position to allow disconnection of the first connector 10A and the second connector 10B in a first position.

In an embodiment, as seen in FIGS. 7B and 7C, the biasing member includes bumps or protuberances 714A, 714B for engaging corresponding grooves provided in the first housing 710. In the first position, the bumps or protuberances 714A, 714B engage the grooves of the first housing 710 to allow the positioning, e.g., connection/disconnection, of the first connector 10A and second connector 10B. For example, the grooves in the first position allow the biasing members 714A of the latch mechanism 714 to be actuated between a first groove and second groove of the first housing 710 so that the projection of the latch mechanism 714 is able to engage or disengage the second connector 10B. In an embodiment, the first housing 710 includes a third bottom groove to allow the latch mechanism 714 to be provided in a second position, in which the latch mechanism 714 is no longer operable, e.g., the latch mechanism 714 can no longer be actuated and prevents the use of the coupling assembly for multiple uses, e.g., single-use. For example, the bottom groove of the first housing 710 includes a sharp undercut, in which after the bump or protuberance 714B has a corresponding structure in which after the bump or protuberance 714B is positioned below the bottom groove, the latch mechanism 714 cannot be further actuated to the engaging/connecting position. That is, when the latch mechanism 714 is pushed downwardly to the second position, and the springs 120, 220 begin to bias the respective valve member 115, 215 so that the second connector 10B is separated from the first connector 10A, the latch mechanism 714 is able to be positioned in the second position to prevent the coupling assembly from any additional use.

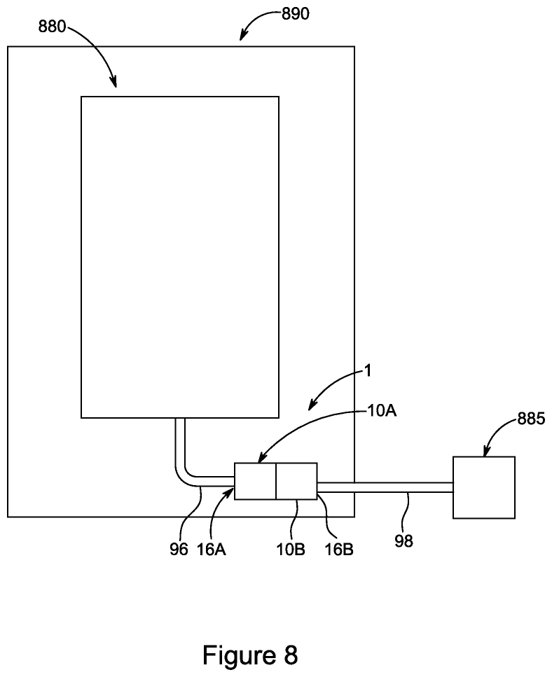

FIG. 8 is a schematic diagram of a bioprocessing bag 880 aseptically connected to a processing device 885 using the assembled aseptic coupling assembly 1. For example, the processing equipment 885 can include equipment that supplies the process material or reactants that form the process material to the fill the bag 880 and/or equipment that utilizes the process material stored within the bioprocessing bag 880. Bioprocessing bag 880 is an example of a fluid storage container. An inlet of the assembly 1 (e.g., opening 16A of the first connector 10A) is fluidly connected to the bioprocessing bag 880 and the outlet (e.g., opening 16B of the second connector 10B) is fluidly connected to the processing equipment 885. For example, the inlet of the assembly 1 is configured to be inserted into tubing 96 that connects to the bioprocessing bag 880, and the outlet of the assembly 1 is configured to be inserted into tubing 98 that connects to the processing equipment 885. In another embodiment, the inlet of assembly 1 (e.g., opening 16A of the first connector 10A) may be configured to be directly attached (e.g., molded, fused, or the like) to the bioprocessing bag 880.

The filled bioprocessing bag 880 is configured to be stored at frozen temperatures (e.g., temperatures of less than 0° C.). For example, the bag holder 890 can be configured to hold the bioprocessing bag 880 within a freezing system (e.g., a blast chiller, low temperature freezer, or the like). In an embodiment, the bioprocessing bag 880 is configured to be stored at a temperature of −50° C. or lower. In an embodiment, the bioprocessing bag 880 is configured to be stored at cryogenic temperatures of −150° C. or lower. In an embodiment, the bioprocessing bag 880 is configured to be stored at cryogenic temperatures of −190° C. or lower. As shown in FIG. 8, the first connector 10A and the second connector 10B are configured so as to be disposable within the bag holder 890 along with the bag 880. For example, the first connector 10A is configured to be disposed in bag holder 890 along with the bag 880 during freezing.

The coupling assembly 1 is configured to form a sealed connection after being frozen and then heated back to ambient temperature with the bioprocessing bag 880. For example, the assembly 1 is configured to provide a sealed connection after being frozen to the storage temperature of the bag 880 and then being heated (e.g., applying heat to the bioprocessing bag 880, exposing the bioprocessing bag 880 to ambient temperature, or the like). The first connector 10A after being frozen with the bioprocessing bag 880 and then being heated back to above freezing (e.g., to ambient temperature) is connected with the second connector 10B to form the sealed connection. In an embodiment, the second connector 10B is not frozen with the first connector 10A and the bag 880. For example, ambient temperature is at or about 20° C. The first connector 10A, the second connector 10B, and the sealing members are formed to have no substantial deformation after being cooled to at least −50° C. then heated to ambient temperature. Substantial deformation includes, for example, visible cracking in the material, a shrinkage or expansion relative to its original shape at ambient temperature that can interfere with the connection of first connector 10A to second connection 10B or adversely affect sealing of said connection. The sealing members are each formed to have no substantial deformation by being made of a material that does remains elastic when cooled even to temperatures such as liquid nitrogen temperatures. In embodiments, suitable sealing members showing no substantial deformation at low temperatures can be determined by testing including, as non-limiting examples, temperature retraction testing and/or brittleness testing. Temperature retraction testing can be performed according to ASTM D1329, ISO 2921, or any other suitable testing methodology for determining suitable retraction properties of the material at temperatures where the sealing members may be used. Brittleness testing can be performed according to ASTM D2137, ISO 28702, or any other suitable testing methodology for determining resistance to cracking at temperatures where the sealing members may be used.

### Aspects

Any of aspects 1-13 can be combined with any of aspects 14-15 and vice versa.

Aspect 1. An aseptic coupling assembly, comprising a first connector including a first fluid passage extending through the first connector, the first fluid passage including an opening, wherein the first connector comprises a first housing having a first end and a second end, a first valve member provided to be slidable within the first housing, and a first spring that biases the first valve member towards the second end of the first housing to a first closed position, a second connector including a second fluid passage extending through the second connector, the second fluid passage including an opening, wherein the second connector comprises a second housing having a first end and a second end, a second valve member provided to be slidable within the second housing, and a second spring that biases the second valve member towards the second end of the second housing to a second closed position, wherein the first valve member and the second valve member include an internal passageway fluidly connected to the first fluid passage and the second fluid passage, respectively, wherein the first connector and the second connector are configured to be coupled together, in which the first valve member is slidable away from the first closed position within the first housing to compress the first spring and the second valve member is slidable away from the second closed position within the second housing to compress the second spring so that the first fluid passage and the second fluid passage are fluidly connected, and wherein the aseptic coupling assembly further comprises a seal that is configured such that fluid flowing through the coupling assembly is prevented from contacting the first spring and the second spring.

Aspect 2. The aseptic coupling assembly of Aspect 1, wherein the first connector further comprises a latch mechanism having a projection and the second connector comprises a groove provided on an outer surface of the second housing such that the projection of the latch mechanism engages the groove to retain the second connector with the first connector when the first connector and the second connector are coupled together.

Aspect 3. The aseptic coupling assembly of Aspect 2, wherein the latch mechanism comprises a biasing member for biasing the latch mechanism in at least one of a retaining position or a displacement position with respect to the first housing of the first connector.

Aspect 4. The aseptic coupling assembly of any of Aspects 1-3, wherein the first valve member and the second valve member each include a slot that fluidly connects the internal passageway of the respective valve member with a space external to the respective valve member.

Aspect 5. The aseptic coupling assembly of Aspect 4, wherein the seal comprises a plurality of sealing members provided between the first connector and the first valve member and the second connector and the second valve member to prevent the fluid flowing through the coupling assembly from contacting the first spring and the second spring.

Aspect 6. The aseptic coupling assembly of Aspect 5, wherein the first valve member and the second valve member each comprise a first sealing member, a second sealing member, and a third sealing member of the plurality of sealing members, wherein the first sealing member is provided between a first end of the valve member and fluid passage of the connector, the second sealing member is provided between the valve member and an internal surface of the housing, and the third sealing member is provided nearer a second end of the valve member than the slot.

Aspect 7. The aseptic coupling assembly of Aspect 6, wherein the third sealing member is configured such that when the respective spring biases the respective valve member towards the second end of the respective housing to the closed position, the third sealing member engages an internally inclined surface of the respective housing to sealingly prevent the fluid from flowing through the respective valve member.

Aspect 8. The aseptic coupling assembly of any of Aspects 1-7, wherein the first spring is provided external to the first fluid passage within the first housing and the second spring is provided external to the second fluid passage within the second housing.

Aspect 9. The aseptic coupling assembly of any of Aspects 1-8, wherein at least one of the first connector and the second connector is formed of a polymer material that does not exhibit cracking and maintains a seal after being cooled to as low as −196° C. then heated back to ambient temperature.

Aspect 10. The aseptic coupling assembly of Aspect 9, wherein at least one of the first connector and the second connector comprises a fluoropolymer.

Aspect 11. The aseptic coupling assembly of Aspect 5, wherein the plurality of sealing members comprises one or more of silicone and ethylene-vinyl acetate (EVA).

Aspect 12. The aseptic coupling assembly of any of Aspects 1-11, wherein the second end of the first housing and the second housing each include a lip portion that extends past the second end of the respective valve member when the respective connector is in the closed position.

Aspect 13. The aseptic coupling assembly of any of Aspects 1-12, wherein at least one of the first connector and the second connector comprises a connection portion that is interchangeable, wherein the connection portion is selected from a luer connection, a hose barb connection, a connection molded to a bag assembly, or a connection that is overmolded to a bag assembly.

Aspect 14. A method of aseptically connecting a fluid storage container, the method comprising: coupling a first connector to a second connector, wherein a first fluid passage extending through the first connector and a second fluid passage extending through the second connector are fluidly connected, the first fluid passage and the second fluidly passage each including an opening, wherein the first connector comprises a first housing having a first end and a second end, a first valve member provided to be slidable within the first housing, and a first spring that biases the first valve member towards the second end of the first housing to a first closed position, and wherein the second connector comprises a second housing having a first end and a second end, a second valve member provided to be slidable within the second housing, and a second spring that biases the second valve member towards the second end of the second housing to a second closed position, wherein coupling the first connector to the second connector includes: engaging a first surface of the first valve member and a first surface of the second valve member such that the first valve member is slid away from the first closed position within the first housing to compress the first spring and the second valve member is slid away from the second closed position within the second housing to compress the second spring so that the first fluid passage and the second fluid passage are fluidly connected; and preventing fluid flowing through the first connector and the second connector from contacting the first spring and the second spring, respectively, by isolating the first spring and the second spring from the fluid.

Aspect 15. The method according to Aspect 14, further comprising disconnecting the first connector and the second connector, wherein during disconnecting the first connector and the second connector the respective spring biases the respective valve member towards the second end of the respective housing to the closed position so that a sealing member engages an internally inclined surface of the respective housing to sealingly prevent the fluid from flowing through the respective valve member

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

What is claimed is:

1. An aseptic coupling assembly, comprising:
a first connector including a first fluid passage extending through the first connector, the first fluid passage including an opening,
wherein the first connector comprises a first housing having a first end and a second end, a first valve member provided to be slidable within the first housing, and a first spring that biases the first valve member towards the second end of the first housing to a first closed position,
a second connector including a second fluid passage extending through the second connector, the second fluid passage including an opening,
wherein the second connector comprises a second housing having a first end and a second end, a second valve member provided to be slidable within the second housing, and a second spring that biases the second valve member towards the second end of the second housing to a second closed position,
wherein the first valve member and the second valve member include an internal passageway fluidly connected to the first fluid passage and the second fluid passage, respectively,
wherein the first connector and the second connector are configured to be coupled together, in which the first valve member is slidable away from the first closed position within the first housing to compress the first spring and the second valve member is slidable away from the second closed position within the second housing to compress the second spring so that the first fluid passage and the second fluid passage are fluidly connected, and
wherein the aseptic coupling assembly further comprises a seal that is configured such that fluid flowing through the coupling assembly is prevented from contacting the first spring and the second spring.

2. The aseptic coupling assembly of claim 1, wherein the first connector further comprises a latch mechanism having a projection and the second connector comprises a groove provided on an outer surface of the second housing such that the projection of the latch mechanism engages the groove to retain the second connector with the first connector when the first connector and the second connector are coupled together.

3. The aseptic coupling assembly of claim 2, wherein the latch mechanism comprises a biasing member for biasing the latch mechanism in at least one of a retaining position or a displacement position with respect to the first housing of the first connector.

4. The aseptic coupling assembly of claim 1, wherein the first valve member and the second valve member each include a slot that fluidly connects the internal passageway of the respective valve member with a space external to the respective valve member.

5. The aseptic coupling assembly of claim 4, wherein the seal comprises a plurality of sealing members provided between the first connector and the first valve member and the second connector and the second valve member to prevent the fluid flowing through the coupling assembly from contacting the first spring and the second spring.

6. The aseptic coupling assembly of claim 5, wherein the first valve member and the second valve member each comprise a first sealing member, a second sealing member, and a third sealing member of the plurality of sealing members, wherein the first sealing member is provided between a first end of the valve member and fluid passage of the connector, the second sealing member is provided between the valve member and an internal surface of the housing, and the third sealing member is provided nearer a second end of the valve member than the slot.

7. The aseptic coupling assembly of claim 6, wherein the third sealing member is configured such that when the respective spring biases the respective valve member towards the second end of the respective housing to the closed position, the third sealing member engages an internally inclined surface of the respective housing to sealingly prevent the fluid from flowing through the respective valve member.

8. The aseptic coupling assembly of claim 1, wherein the first spring is provided external to the first fluid passage within the first housing and the second spring is provided external to the second fluid passage within the second housing.

9. The aseptic coupling assembly of claim 1, wherein at least one of the first connector and the second connector is formed of a polymer material that does not exhibit cracking and maintains a seal after being cooled to as low as −196° C. then heated back to ambient temperature.

10. The aseptic coupling assembly of claim 9, wherein at least one of the first connector and the second connector comprises a fluoropolymer.

11. The aseptic coupling assembly of claim 5, wherein the plurality of sealing members comprises one or more of silicone and ethylene-vinyl acetate (EVA).

12. The aseptic coupling assembly of claim 1, wherein the second end of the first housing and the second housing each include a lip portion that extends past the second end of the respective valve member when the respective connector is in the closed position.

13. The aseptic coupling assembly of claim 1, wherein at least one of the first connector and the second connector comprises a connection portion that is interchangeable, wherein the connection portion is selected from a luer connection, a hose barb connection, a connection molded to a bag assembly, or a connection that is overmolded to a bag assembly.

14. A method of aseptically connecting a fluid storage container, the method comprising:
coupling a first connector to a second connector, wherein a first fluid passage extending through the first connector and a second fluid passage extending through the second connector are fluidly connected, the first fluid passage and the second fluidly passage each including an opening, wherein the first connector comprises a first housing having a first end and a second end, a first valve member provided to be slidable within the first housing, and a first spring that biases the first valve member towards the second end of the first housing to a first closed position, and wherein the second connector comprises a second housing having a first end and a second end, a second valve member provided to be slidable within the second housing, and a second spring that biases the second valve member towards the second end of the second housing to a second closed position,
wherein coupling the first connector to the second connector includes:
engaging a first surface of the first valve member and a first surface of the second valve member such that the first valve member is slid away from the first closed position within the first housing to compress the first spring and the second valve member is slid away from the second closed position within the second housing to compress the second spring so that the first fluid passage and the second fluid passage are fluidly connected; and
preventing fluid flowing through the first connector and the second connector from contacting the first spring and the second spring, respectively, by isolating the first spring and the second spring from the fluid.

15. The method according to claim 14, further comprising disconnecting the first connector and the second connector, wherein during disconnecting the first connector and the second connector the respective spring biases the respective valve member towards the second end of the respective housing to the closed position so that a sealing member engages an internally inclined surface of the respective housing to sealingly prevent the fluid from flowing through the respective valve member.
